(19) United States
(12) Patent Application Publication
(10) Pub. No.: US 2026/0098267 A1
(43) Pub. Date: Apr. 9, 2026

(54) Treating herpesvirus-mediated intestinal dysfunction for prevention of age-related neurodegeneration

(71) Applicant: Battle Biotech, LLC, Ypsilanti, MI (US)

(72) Inventor: Richelle Gayle Cutler, Ypsilanti, MI (US)

(21) Appl. No.: 19/346,483

(22) Filed: Sep. 30, 2025

Related U.S. Application Data
(63) Continuation of application No. 16/709,607, filed on Dec. 10, 2019, now Pat. No. 12,428,643.

(51) Int. Cl. C12N 15/113; A61K 9/00; A61K 45/06; C12N 1/20

(52) U.S. Cl. CPC C12N 15/1133; C12N 1/20; A61K 9/0053; A61K 45/06; C12N 2310/122; C12N 2310/141

(57) ABSTRACT

Human herpesviruses can infect barrier and immune cells of the intestinal tract to cause microbiome dysbiosis and inflammation. Microbiome dysbiosis can affect the availability of nutrients required for normal brain function. Inflammation can compromise the intestinal barrier and lead to microbial translocation. Microbial translocation can cause brain infection or a mimicry auto-immune response. The embodiments restrict herpesvirus activity and injury by 1) the oral administration of exosomes containing factors to inhibit viral activity and restore homeostasis, and 2) the oral administration of recombinant bacteria that produce exosomes containing factors to inhibit viral activity and restore homeostasis. Another embodiment is the treatment of alpha-HHVs mediated intestinal dysfunction by nucleoside analogs, such as famciclovir, which may be combined with anti-viral exosome therapy.

### REFERENCE TO PRIORITY APPLICATION

This application claims benefit of the filing date of U.S. Non-Provisional patent application Ser. No. 16/709,607, filed on Dec. 10, 2019, the contents of which are incorporated herein in its entirety.

### REFERENCE TO THE SEQUENCE LISTING

The instant application contains a sequence listing which is herein submitted electronically in ST.26 format and hereby incorporated by reference in its entirety. Said sequence listing, 10001B-US-CON-ST.26, was created on Sep. 30, 2025, and is 308,264 bytes in size.

### BACKGROUND

Infection with one or more human herpesviruses (HHVs) is ubiquitous in most populations by the eighth decade of life. HHVs primarily enter the body through trigeminal ganglia or olfactory neurons. Virions shed from these neurons can enter circulating monocytes and dendritic precursors. Circulating monocytes and dendritic precursors differentiate when they encounter inflammatory cytokines. Phagocyte and dendritic cell differentiation reactivates HHV from latency. Apart from replicating reservoirs, HHVs mostly remain latent through the first seven decades. The frequency of HHV reactivation depends on the HHV strain and viral load, and host variables such as stress, age, and genetics. For instance, human cytomegalovirus (HCMV) is an HHV that remains latent but reactivates in individuals experiencing stress, depressed immunity or in healthy individuals over the age of 70. Genetic variants, such as polymorphisms in type 3 interferon, (IFNL3/4) affect susceptibility to HCMV activation.

There are eight known HHVs and each can infect cells residing in the intestinal wall or tract. HCMV (HHV5) replicates in epithelial, fibroblast, endothelial, and dendritic cells. The Epstein-Barr virus (EBV) infects B lymphocytes. The roseolovirus (HHV6/7) causes Roseola infantum, and replicates in peripheral blood mononuclear cells (PBMC), NK cells, and T lymphocytes. Herpes simplex virus (HSV) and varicella zoster virus (VZV) replicate in fibroblast, epithelial cells, and sensory neurons.

HHV infections could contribute to developing Alzheimer's disease, Parkinson's disease, Lewy body dementia, vascular dementia, frontotemporal dementia, and amyotrophic lateral sclerosis. A causal relationship between HHV infection and age-related neurodegeneration remains unproven. Researchers have found no HHV association with age-neurodegenerations, and those that have reported an association have not proven causation. Interestingly, congenital HCMV infections with severe brain degeneration show a conspicuous absence of viral DNA, which suggests that a non-cell autonomous bystander effect mediates significant pathology. This could explain why some studies failed to find a difference in HHV between brains from control and neurodegeneration patients. The scenario is reminiscent of the HIV-associated neurocognitive disorder mediated by the HIV protein TAT. Association by seropositivity is also problematic. HCMV seronegative subjects can test PCR positive for HCMV DNA in blood, and seropositive subjects can consistently test PCR negative for HCMV DNA. Hcmv can also reactivate intermittently in short intervals, so blood HCMV DNA might be negative one week and positive the next. Thus, researchers may have erroneously concluded that no association exists between some HHVs and age-related neurodegeneration.

Furthermore, HHVs, like HCMV, infect immune cells, and their infection status is associated with oxidative stress, shortened telomeres of lymphocytes, reduced B cell lineages, and T-cell senescence. The subsequent effects of HHV infection on immunity may increase host susceptibility to other opportunistic infections, which are associated with age-related neurodegeneration. Impaired immunity may affect the diversity of symbiotic gut microbiota. Indeed, Alzheimer's disease patients have reduced microbial diversity. A human host utilizes factors manufactured by gut bacteria. For instance, some bacterial strains produce gamma-aminobutyric acid (GABA), which is thought to be transported to the brain. GABA is an inhibitory neurotransmitter reported to be deficient in brains of Alzheimer's disease patients. Accordingly, the pathogenesis of some neurodegenerations may not originate in the brain.

### SUMMARY OF THE INVENTION

Alteration in gut microbiota and gastrointestinal tract inflammation can have deleterious health effects. Human herpesvirus infection of cells related to gastrointestinal system can cause intestinal inflammation, microbiota dysbiosis, barrier dysfunction, microbial-translocation, and neurodegeneration. The invention describes treatments for preventing or treating intestinal dysfunction caused by human herpesviruses by the oral delivery of exosomes, or the ingestion of recombinant bacteria engineered to release therapeutic factors in exosomes. Treatments such as described will at last provide therapy to prevent, delay, or cure age-related neurodegenerations derived from an unsuspected cause, intestinal herpesvirus infection.

### DETAILED DESCRIPTION

In addition to the HHV association noted in the Background, age-related neurodegeneration is associated with gut microbiota dysbiosis. HHV infection of B lymphocytes, T-cells, dendritic cells, and PBMCs can compromise intestinal immunity and increase pathogen-mediated inflammation. Increased inflammation is associated with gut microbiota dysbiosis, where the prevalence of healthy symbiotic bacteria is decreased, and pathogenic bacteria increased. Accordingly, HHV infection is associated with colitis and exacerbated ulcerative colitis in healthy immunocompetent patients. Age-related neurodegeneration has also been associated with genetic variants affecting innate immunity and microbial agents. HHV infection of intestinal epithelial cells, vascular endothelial cells, and fibroblasts can compromise the intestinal barrier and allow microbial translocation.

HHV infection can cause oxidative stress that may accelerate stem cell senescence over time. Additionally, HHV is associated with impaired the Wnt/β-catenin signaling and loss of β-catenin-mediated gene transcription; β-catenin is required for epithelial cell specification, maintenance, and replication. Replicative senescence of transiently replicating epithelium and stem cells can decrease barrier function. Decreased barrier function, often called “leaky gut”, can permit microbial translocation. The elderly with reduced immunity and or compromised blood-brain-barrier are at increased risk for microbes entering the brain. Microbial infection in the brain can initiate an inflammatory response that causes age-related neurodegeneration. Further, with advanced age, the inflammatory response is more sustained. Inflammatory bowel diseases, such as Crohn's disease and ulcerative colitis, are associated with a leaky gut, and accordingly, arc associated with an increased rate of Parkinson's disease. Genetic variants that increase the risk of Crohn's disease are also associated with Parkinson's disease. Mice injected with gut bacteria from Parkinson's disease patients develop neuronal α-synuclein clumping and behavioral symptoms supporting a microbial translocation etiology of Parkinson's disease. Further support of a gut-related etiology for Parkinson's disease comes from the presence of α-synuclein aggregates within the enteric nerve terminals of every examined Parkinson's disease patient. Compared to healthy age-matched controls, Parkinson's disease patients also have reduced occludin protein, a component of tight junctions which could disrupt the intestinal epithelial barrier.

Parkinson's disease patients have significantly more serum HSV IgG antibodies compared to controls, P<0.001. HSV1 DNA was found in the celiac ganglia of a transplant patient and detected in the ganglia innervating the gastrointestinal tract in eight out of ten subjects. In mouse models of enteric HSV1 infection, macrophages-recruited to HSV1 infected neurons release reactive nitrogen species causing neuron injury. Injury to enteric plexus neurons by HHV-infection can explain the most frequent symptom of Parkinson's disease patients, constipation.

A variety of translocated microbes could explain the heterogenous nature of age-related neurodegeneration. Microbe phagocytosis by antigen-presenting cells selects an immunogen to initiate an immune response. If a host protein has a similar sequence to a microbial epitope, it will cross-react and cause autoimmune disease by mimicry. For instance, a UniProt blast of tau protein against bacterial proteasomes identified bacteria with high sequence similarity to proteins from bacterial strains of Actinomyces, Streptococcus, and Streptomyces. The α-synuclein protein, which forms aggregates in Parkinson's disease patients, has sequence-similarity to proteins found in fecal bacteria. A-UniProt blast of α-synuclein against microbial protcomes identified sequence similarity with the uncharacterized protein from the Verrucomicrobia bacterium, with 43.7% positives (2.3e-04 E-value), and with similarity to the chromosome segregation ATPase from Bifidobacterium, with 47% positives (1.4c-03 E-value).

Anti-α-synuclein IgG2 is significantly increased in the plasma from Parkinson's disease patients. IgG2 increases in response to bacterial infections and reacts to bacterial polysaccharides. Intracellular antibodies can cause target aggregation, which are both degraded by the proteasome. Proteasome degradation slows with aging, which could explain age-related onset of age-related neurodegeneration. It is not known whether IgG physically associates with age-related neurodegeneration related-protein aggregates. However, levels of antinuclear antibodies increase with age and are associated with signs of senescence, e.g., telomere shortening. Antinuclear antibodies, indicative for autoimmune disease, are frequently associated with herpes simplex encephalitis.

A chronic subclinical HCMV infection in the colon could impair barrier function and allow microbial translocation. Interestingly, Parkinson's disease is associated with significantly less CD8+ T cell senescence compared to age-matched control, which could result if CD8+ T cells do not engage in restricting viral pathogens. Thus, while Parkinson's disease patients can mount an effective cytotoxic CD8+T immune response against invading microbes, activation requires professional antigen-presenting cells, such as dendritic cells, which may be functionally compromised by HCMV infection.

It is highly likely that enteric sensory neurons harbor HSV and VZV, like the olfactory and trigeminal neurons, and that their reactivation contributes to intestinal inflammation and microbiota dysbiosis. Preventing intestinal HHV activity in intestinal cells can prevent inflammation, microbiota dysbiosis, barrier dysfunction, autoimmune disease, and subsequent age-related neurodegeneration. HSV and VZV infection are treated with nucleoside analogs acyclovir and penciclovir.

There is a long-sought need for an effective Parkinson's disease treatment. Treatment of Parkinson's disease with anti-alpha-HHV nucleoside analogs is not obvious. Unfortunately, long term use of nucleoside analogs can result in HSV and VZV thymidine kinase mutations that render the virus nucleoside resistant. Moreover, HCMV antiviral treatments have serious side-effects, develop resistance, and are not long-term options. A novel method for preventing intestinal HHV-activity through oral delivery exosomes containing stock and customized antiviral factors. Exosomes can be isolated by ultracentrifugation or serial filtration from modified human colonic fibroblast (ATCC-49). Exosomes from colonic fibroblast may contain therapeutic factors for intestinal epithelial cells. For instance, exosomes from a colon fibroblast cell line carry amphiregulin, which binds epidermal growth factor receptor, and rescues intestinal epithelial cells from cell death in organoid culture. HCMV-infected cultured human fibroblasts have markedly altered miRNA expression. Thus, in situ HCMV-infected intestinal niche fibroblasts are likely to have altered exosome content, which may be deleterious to epithelial maintenance. Exosome content from cultured fibroblasts can be customized by inserting donor DNA that encodes pre-miRNAs, mRNA, or long non-coding RNA (lncRNA) into the fibroblast genome using the Sleeping Beauty (SB) transposon system. Construct expression may be driven by fibroblast housekeeping promoters.

Exogenous exosomes might need to penetrate through mucosal bacteria to enter epithelial cells, vascular endothelial cells, and their respective stem cells. If exosomes do not reach these cells, HHV-activity may be unaffected in these cells. Exosome transfer occurs between the intestinal epithelial cells and bacteria. A novel method to prevent and treat HHV-mediated inflammation, microbiota dysbiosis, barrier dysfunction, autoimmune disease, and subsequent age-related neurodegeneration is to ingest recombinant bacteria that will survive among the gut flora and release exosomes containing antiviral factors. The type of bacteria selected is critical to therapeutic efficacy. The bacteria Clostridium, Lactobacillus, Enterococcus, and Akkermansia are associated with the mucosal surface, and therefore have the proximity to deliver high concentrations of exosomes with antiviral factors to intestinal cells. The selected bacterial strains may have high genetic tractability. Bacteria are bioengineered to express constructs encoding miRNA, mRNA, and other oligonucleotides using modern techniques for DNA transformation, stable integration by site-specific recombination, and selection. Expression constructs may include coding to direct factors to sort into exosomes for secretion.

In some embodiments, expression constructs are designed to express select Ex-miRNA/shRNA, mRNA, lncRNA and protein as exosome cargo that reduce HHV activity, oxidative stress, inflammation, and increase replicative capacity of intestinal epithelial and vascular endothelial cells. The following are examples of factors delivered in exosomes. In patients with increased HCMV activity, exosomes could include HCMV-miRNAs that suppress active replication. Human antiviral miRNAs proven to reduce HHV replication include miR-324-5p (SEQ ID NO: 1), miR-185 (SEQ ID NO: 2), miR-29b (SEQ ID NO: 3), miR-1287 (SEQ ID NO: 4), miR-199a-3p (SEQ ID NO: 5), miR-214 (SEQ ID NO: 6), miR-21 (SEQ ID NO: 7), miR-200b-3p (SEQ ID NO: 8), miR-200c-3p (SEQ ID NO: 9), miR-221 (SEQ ID NO: 10) and miR-24-1 (SEQ ID NO: 11). Proteins involved in antiviral defense, such as Sp100 encoded by SEQ ID NO: 12, Daxx encoded by SEQ ID NO: 13, PML encoded by SEQ ID NO: 14, BclAF1 encoded by SEQ ID NO: 15, Tetherin/Bts-2 encoded by SEQ ID NO: 16, Trim 5 alpha encoded by SEQ ID NO: 17, and Apobec-3G encoded by SEQ ID NO: 18, could be delivered as mRNA or protein. For instance, Daxx directs HDAC to the major immediate early gene promoter to silence viral transcription, and BclAF1 is critical for interferon 1 response; both proteins are degraded by HHVs.

An embodiment includes delivering ApoE2/3-ch in exosome. Aside from the autosomal dominant mutations causing early-onset AD, apolipoprotein E (ApoE) allelic variants explain the highest variance in AD risk. The ApoE4 allele confers the greatest AD risk, with ApoE3 neutral, and ApoE2, a protective variant. An explanation for higher AD risk with ApoE4 risk could be related to higher intracellular cholesterol content, especially in lipid rafts, which would increase virion binding affinity. Higher intercellular cholesterol would also provide ample cholesterol for virion assembly. Another explanation is that ApoE2/E3 has less affinity for LDL or LDLR than ApoE4 and may be more available to bind viral glycoproteins or cholesterol both inside and outside the cell. Accordingly, the LDLR and LDL binding domains of ApoE3 can restrict HIV infectivity. The LDLR domain can even protect against early-onset AD from the autosomal dominant mutation, PSEN1, in patients homozygous for the ApoE3-Christchurch mutation (R136S). Moreover, ApoE2/3 is more concentrated in serum than ApoE4. Therefore, delivering ApoE2, for example the sequence encoded by SEQ ID NO: 19 or ApoE3-Christchurch protein for example the sequence encoded by SEQ ID NO: 20, or LDLR domain to intestinal cells via exosomes may protect against HHV infectivity.

An embodiment includes short hairpin RNAs (shRNA) artificially designed to silence HHV gene expression. For instance, a shRNA targeting the HCMV UL83 gene (pp65) at the sequence GCAAGATCTCGCACATCATGC (SEQ ID NO: 21) will reduce pp65 protein expression and release its inhibition on innate and adaptive immunity. Targeting the smallest capsid protein at the sequence GCGCATGTCCAGTCTGTTTAA (SEQ ID NO: 22) can reduce HCMV yield by 10,000-fold. Additional shRNAs designed to restrict HHV gene expression can be included in the fibroblast and bacteria expression constructs.

Another embodiment includes treating the oxidative stress caused by the HHV. For instance, HCMV increases NAD (P) H oxidase (NOX) activity in endothelial cells, and NOX4 expression is increased in vascular endothelial cells from Alzheimer's disease patients. NOX4 expression is inhibited by miR-137 (SEQ ID NO: 23) and miR-99a (SEQ ID NO: 24). Messenger RNAs encoding proteins that reduce oxidative stress include superoxide dismutase encoded by SEQ ID NO: 25, glutathione reductase encoded by SEQ ID NO: 26, and the protein Hic-5 encoded in SEQ ID NO: 27, which inhibits NOX4 activity by promoting the ubiquitin-proteasome degradation of NOX4.

Another embodiment includes reducing intestinal inflammation by delivering exosome with anti-inflammatory factors. For instance, miR-219a-5p (SEQ ID NO: 28) delivered to T cells downregulates Th1/Th17 cell differentiation. Delivery of miR-146a (SEQ ID NO: 29), miR-19b (SEQ ID NO: 30), miR-590-5p (SEQ ID NO: 31) and miR-495 (SEQ ID NO: 32) may reduce inflammatory cell injury. Delivery of the lncRNA, Mirt2 (SEQ ID NO: 33), to dendritic cells upregulates IL-22 expression and helps resolve chronic inflammation. Macrophages that sense HHV release nitric oxide and inadvertently injure neurons. The microRNA-146a (SEQ ID NO: 29) reduces nitric oxide synthase expression and will reduce nitric oxide release from macrophages.

Another embodiment includes extending the replicative capacity of epithelial and vascular endothelial cells. For instance, HCMV may control replicative senescence of epithelial and endothelial cells. For instance, HCMV infection increases DKK1 expression. DKK1 interacts with Wnt co-receptors LRP5/6, leading to the degradation of β-catenin. Degradation of β-catenin prevents Wnt pathway signaling required for epithelial cell homeostasis and renewal. EBV miRNA, miR-BART10-3p, reduces DKK1 protein expression by 60%. To prevent oncogenic potential, an inducible promoter can be used to drive miR-BART10-3p (SEQ ID NO: 34) expression in recombinant bacteria.

To ensure that miRNAs, mRNA, lncRNA, and protein are sorted to the exosome, the donor DNA may include a 3′ export motif, GGAG or GGCU, for hnRNPA2B1 and SYNCRIP processing respectively. Messenger RNA encoding protein may be directed to the exosome by including a fused ubiquitylated-coding sequence (CTGCC) for ECSRT-dependent processing or a CD63 association sequence for ECSRT-independent processing.

Exosomes from cultured fibroblasts and recombinant bacteria may be isolated by ultracentrifugation or serial filtration to characterize content. Exosome cargo will be tested for the ability to reduce HHV-activity, oxidative stress, inflammation, and induce replication in columnar intestinal epithelial cell cultures. With optimal exosome cargo defined, cultivation of recombinant fibroblast or recombinant bacteria will be scaled for mass production.

HHVs reactivate during immunosuppression, such as with transplant or chemotherapy, to cause life-threatening diseases. The described novel treatments are useful for restricting HHV-mediated intestinal disease and may be administered prophylactically in immunosuppressed patients.

1. A method of preventing or treating human herpesvirus infection-mediated inflammation in the gastrointestinal tract of a subject by oral administration of exosomes contain miRNA, mRNA, and a combination thereof.

2. The method according to claim 1, wherein the human herpesvirus-infection is intestinal.

3. The method according to claim 1, wherein the inflammation is derived from human herpesvirus type selected from the group consisting of human cytomegalovirus, herpes simplex virus 1, herpes simplex virus 2, Epstein-Barr virus, varicella zoster virus, roseolovirus (HHV6, HHV7), Kaposi sarcoma virus (HHV8), and a combination thereof.

4. The method according to claim 1, wherein the exosome includes mRNAs encoding anti-inflammatory proteins selected from superoxide dismutase (SEQ ID NO:25), glutathione reductase (SEQ ID NO:26), APOE2 (SEQ ID NO: 19), APOE3-christchurch (SEQ ID NO: 20), and a combination thereof.

5. A method of preventing or treating human herpesvirus infection-mediate inflammation in the gastrointestinal tract of a subject, the method comprising the oral administration of recombinant gram-negative bacteria engineered to contain a genome-integrated expression construct for expression of anti-inflammatory protein, miRNA, or both, that are exported from bacteria in vesicles.

6. The method according to claim 5, wherein the recombinant bacteria are from the genus Akkermansia.

7. The method according to claim 5, wherein the human herpesvirus type is selected from the group consisting of human cytomegalovirus, herpes simplex virus 1, herpes simplex virus 2, Epstein-Barr virus, varicella zoster virus, roseolovirus (HHV6, HHV7), HHV8, and a combination thereof.

8. The method according to claim 5, wherein the expression construct is under the control of an inducible promoter.

9. The method according to claim 5, wherein the genome-integrated expression construct encodes mRNA translated into anti-inflammatory protein selected superoxide dismutase (SEQ ID NO:25), glutathione reductase (SEQ ID NO:26), APOE2 (SEQ ID NO: 19), APOE3-christchurch (SEQ ID NO: 20), and a combination thereof.

10. The method of claim 1, wherein the anti-inflammatory miRNA are selected from the group consisting of hsa-miR-324 (SEQ ID NO: 1), hsa-miR-185 (SEQ ID NO: 2), hsa-mir-29b-1 (SEQ ID NO: 3), hsa-mir-221 (SEQ ID NO: 10), hsa-miR-24-1 (SEQ ID NO: 11), hsa-miR-137 (SEQ ID NO: 23), hsa-miR-99a (SEQ ID NO: 24), hsa-miR-219a (SEQ ID NO: 28), hsa-miR-146 (SEQ ID NO: 29), hsa-miR-19b (SEQ ID NO: 30), hsa-miR-495 (SEQ ID NO: 32), and a combination thereof.

11. The method of claim 5, wherein the genome-integrated expression construct encodes anti-inflammatory miRNA selected from the group consisting of hsa-miR-324 (SEQ ID NO: 1), hsa-miR-185 (SEQ ID NO: 2), hsa-mir-29b-1 (SEQ ID NO: 3), hsa-mir-221 (SEQ ID NO: 10), hsa-miR-24-1 (SEQ ID NO: 11), hsa-miR-137 (SEQ ID NO: 23), hsa-miR-99a (SEQ ID NO: 24), hsa-miR-219a (SEQ ID NO: 28), hsa-miR-146 (SEQ ID NO: 29), hsa-miR-19b (SEQ ID NO:30), hsa-miR-495 (SEQ ID NO: 32), and a combination thereof.

12. The method of claim 10, wherein the exosomes further contain herpesvirus antiviral shRNA that silences human herpesvirus gene expression.

13. The method of claim 11, wherein the genome-integrated expression constructs further encodes herpesvirus antiviral shRNA that silences human herpesvirus gene expression.

14. The method of claim 10, wherein the exosomes further contain a shRNA targeting human cytomegalovirus pp65 mRNA (SEQ ID NO: 21), and/or a shRNA targeting the human cytomegalovirus smallest capsid protein mRNA (SEQ ID NO: 22).

15. The method of claim 11, wherein the expression construct further encodes a shRNA targeting human cytomegalovirus pp65 mRNA (SEQ ID NO: 21), and/or a shRNA targeting the human cytomegalovirus smallest capsid protein mRNA (SEQ ID NO: 22).

16. The method of claim 10, wherein the exosomes further contain herpesvirus antiviral miRNA selected from a group consisting of hsa-miR-185 (SEQ ID NO: 2), hsa-miR-199a (SEQ ID NO: 5), hsa-miR-200b (SEQ ID NO: 8), hsa-miR-200c (SEQ ID NO:9), hsa-miR-219a (SEQ ID NO: 28), and a combination thereof.

17. The method of claim 11, wherein the expression construct further encodes antiviral miRNA selected from a group consisting of hsa-miR-185 (SEQ ID NO: 2), hsa-miR-199a (SEQ ID NO: 5), hsa-miR-200b (SEQ ID NO:8), hsa-miR-200c (SEQ ID NO: 9), hsa-miR-219a (SEQ ID NO: 28), and a combination thereof.


SEQUENCE LISTING (37 sequences)

SEQ ID NO: 1 - RNA, 22 nt, Homo sapiens
  source 1..22: mol_type = other RNA
cgcatcccctagggcattggtg

SEQ ID NO: 2 - RNA, 63 nt, Homo sapiens
  source 1..63: mol_type = other RNA
gggggcgagggaggagagaaaggcagccgaggccccccccaggggcggccccggcccccccca

SEQ ID NO: 3 - RNA, 80 nt, Homo sapiens
  source 1..80: mol_type = other RNA
ttcaggaagctggtttcatatggtggtttagatttaaatagtgattgtctagcaccatttgaaatcagtgttcttggggg

SEQ ID NO: 4 - RNA, 90 nt, Homo sapiens
  source 1..90: mol_type = other RNA
gttgtgctgtccaggtgctggatcagtggttcgagtctgagcctttaaaagccactctagccacagatgcagtgattggagccatgacaa

SEQ ID NO: 5 - RNA, 22 nt, Homo sapiens
  source 1..22: mol_type = other RNA
acagtagtctgcacattggtta

SEQ ID NO: 6 - RNA, 109 nt, Homo sapiens
  source 1..109: mol_type = other RNA
gcctggctggacagagttgtcatgtgtctgcctgtctacacttgctgtgcagaacatccgctcacctgtacagcaggcacagacaggcagtcacatgacaacccagcct

SEQ ID NO: 7 - RNA, 72 nt, Homo sapiens
  source 1..72: mol_type = other RNA
tgtcgggtagcttatcagactgatgttgactgttgaatctcatggcaacaccagtcgatgggctgtctgaca

SEQ ID NO: 8 - RNA, 22 nt, Homo sapiens
  source 1..22: mol_type = other RNA
taatactgcctggtaatgatga

SEQ ID NO: 9 - RNA, 23 nt, Homo sapiens
  source 1..23: mol_type = other RNA
taatactgccgggtaatgatgga

SEQ ID NO: 10 - RNA, 109 nt, Homo sapiens
  source 1..109: mol_type = other RNA
gaacatccaggtctggggcatgaacctggcatacaatgtagatttctgtgttcgttaggcaacagctacattgtctgctgggtttcaggctacctggaaacatgttctc

SEQ ID NO: 11 - RNA, 68 nt, Homo sapiens
  source 1..68: mol_type = other RNA
ctccggtgcctactgagctgatatcagttctcattttacacactggctcagttcagcaggaacaggag

SEQ ID NO: 12 - RNA, 2118 nt, Homo sapiens
  source 1..2118: mol_type = mRNA
ctccggtgcctactgagctgatatcagttctcattttacacactggctcagttcagcaggaacaggagagacgctgtggtctcacctgtcctggcaaggggcctctgccggctgttcccatgactggctcagggtctgagttcttattccatcaaccttgatcaaaagaaggaaagggaagaaaaaggcccagggaggctgaatgaatgtatttcaccagtagcaaatgagatgaaccatcttcctgcacacagccacgatttgcaaaggtttttggagaggaataaatttaatgaaagatgtacatgacttctaaaaactataagcagtgctgggtaaaattaaacacatgatgttcacggaagaccagggtgtagatgacaggctgctctatgacattgtattcaagcacttcaaaagaaataaggtggagatttcaaatgcaataaaaaagacatttccattcctcgagggcctccgtgatcgtgatctcatcacaaataaaatgtttgaagattctcaagattcttgtagaaacctggtccctgtacagagagtggtgtacaatgttcttagtgaactggagaagacatttaacctgccagttctggaagcactgttcagcgatgtcaacatgcaggaataccccgatttaattcacatttataaaggctttgaaaatgtaatccatgacaaattgcctctccaagaaagtgaagaagaagagagggaggagaggtctggcctccaactaagtcttgaacaaggaactggtgaaaactcttttcgaagcctgacttggccaccttcgggttccccatctcatgctggtacaaccccacctgaaaatggactctcagagcacccctgtgaaacagaacagataaatgcaaagagaaaagatacaaccagtgacaaagatgattcgctaggaagccaacaaacaaatgaacaatgtgctcaaaaggctgagccaacagagtcctgcgaacaaattgctgtccaagtgaataatggggatgctggaagggagatgccctgcccgttgccctgtgatgaagaaagcccagaggcagagctacacaaccatggaatccaaattaattcctgttctgtgcgactggtggatataaaaaaggaaaagccattttctaattcaaaagttgagtgccaagcccaagcaagaactcatcataaccaggcatctgacataatagtcatcagcagtgaggactctgaaggatccactgacgttgatgagcccttagaagtcttcatctcagcaccgagaagtgagcctgtgatcaataatgacaaccctttagaatcaaatgatgaaaaggagggccaagaagccacttgctcacgaccccagattgtaccagagcccatggatttcagaaaattatctacattcagagaaagttttaagaaaagagtgataggacaagaccacgacttttcagaatccagtgaggaggaggcgcccgcagaagcctcgagcggggcactgagaagcaagcatggtgagaaggctcctatgacttctagaagtacatctacttggagaatacccagcaggaagagacgtttcagcagtagtgacttttcagacctgagtaatggagaagagcttcaggaaacctgcagctcatccctaagaagagggtcaggtaaagaagattaggatgccaagacttggcctgcagaatgtcaggaatgtgaattaaaagctgctgtttccagacgctttttattctgagcaccttcactaccttgtatccagttcatctgggaactcctttttgcattttagaaaatggaaagaggcaggaaattatgataaactcatgtttaacagaaagagtttcactgactaaatgtatgtaattatattttgttgttgtagaagaaataaatagcaaatttgtggtattcttttttttaaacctgctctcattcctattaacactaagatcttagatttttatagtgataaatgggttgacatcattgtcatttgtaattgtaaagcctcaaaagacaactgttcctactatgtaattatagacagaaataaaaacttcagatcaaacactctcaaacgttaaaaaaaaa

SEQ ID NO: 13 - DNA, 2223 bp, Homo sapiens
  source 1..2223: mol_type = genomic DNA
atggccaccgctaacagcatcatcgtgctggatgatgatgacgaagatgaagcagctgctcagccagggccctcccacccactccccaatgcggcctcacctggggcagaagcccctagctcctctgagcctcatggggccagaggaagcagtagttcgggcggcaagaaatgctacaagctggagaatgagaagctgttcgaagagttccttgaactttgtaagatgcagacagcagaccaccctgaggtggtcccattcctctataaccggcagcaacgtgcccactctctgtttttggcctcggcggagttctgcaacatcctctctagggtcctgtctcgggcccggagccggccagccaagctctatgtctacatcaatgagctctgcactgttctcaaggcccactcagccaaaaagaagctgaacttggcccctgccgccaccacctccaatgagccctctgggaataaccctcccacacacctctccttggaccccacaaatgctgaaaacactgcctctcagtctccaaggacccgtggttcccggcggcagatccagcgtttggagcagctgctggcgctctatgtggcagagatccggcggctgcaggaaaaggagttggatctctcagaattggatgacccagactccgcatacctgcaggaggcacggttgaagcgtaagctgatccgcctctttgggcgactatgtgagctgaaagactgctcttcactgaccggccgtgtcatagagcagcgcatcccctaccgtggcacccgctacccagaggttaacaggcgcattgagcggctcatcaacaagccagggcctgataccttccctgactatggggatgtgcttcgggctgtagagaaggcagctgcccgacacagccttggcctcccccgacagcagctccagctcatggctcaggatgccttccgagatgtgggcatcaggttacaggagcgacgtcacctcgatctcatctacaactttggctgccacctcacagatgactataggccaggcgttgaccctgcactatcagatcctgtgttggcccggcgccttcgggaaaaccggagtttggccatgagtcggctggatgaggtcatctccaaatatgcaatgttgcaagacaaaagtgaggagggcgagagaaaaaagagaagagctcggctccaaggcacctcttcccactctgcagacacccccgaagcctccttggattctggtgagggccctagtggaatggcatcccaggggtgcccttctgcctccagagctgagacagatgacgaagacgatgaggagagtgatgaggaagaggaggaggaggaggaagaagaagaggaggaggccacagattctgaagaggaggaggatctggaacagatgcaggagggtcaggaggatgatgaagaggaggacgaagaggaagaagcagcagcaggtaaagatggagacaagagccccatgtcctcactacagatctccaatgaaaagaacctggaacctggcaaacagatcagcagatcttcaggggagcagcaaaacaaaggacgcatagtgtcaccatcgttactgtcagaagaacccctggccccctccagcatagatgctgaaagcaatggagaacagcctgaggagctgaccctggaggaagaaagccctgtgtctcagctctttgagctagagattgaagctttgcccctggataccccttcctctgtggagacggacatttcctcttccaggaagcaatcagaggagcccttcaccactgtcttagagaatggagcaggcatggtctcttctacttccttcaatggaggcgtctctcctcacaactggggagattctggtcccccctgcaaaaaatctcggaaggagaagaagcaaacaggatcagggccattaggaaacagctatgtggaaaggcaaaggtcagtgcatgagaagaatgggaaaaagatatgtaccctgcccagcccaccttcccccttggcttccttggccccagttgctgattcctccacgagggtggactctcccagccatggcctggtgaccagctccctctgcatcccttctccagcccggctgtcccaaaccccccattcacagcctcctcggcctggtacttgcaagacaagtgtggccacacaatgcgatccagaagagatcatcgtgctctcagactctgattaa

SEQ ID NO: 14 - RNA, 2251 nt, Homo sapiens
  source 1..2251: mol_type = mRNA
gccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcggtctcttccagccctcagtctgaggttctgtattggaaagtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccagga

SEQ ID NO: 15 - RNA, 4373 nt, Homo sapiens
  source 1..4373: mol_type = mRNA
ttcctcgcgttcttgagtcgggaaatggccgctgtgtggttgcaacggagataaattcccggaaccgcgattcggcgtgtcaggaattcgaatttagagtttaatttctcagagcattctctccaggaagaatttttacagtatctcaaagacttcacttgacttcttgatcctgcataaaaccaaggagaaaagaaatgggtcgctccaattctagatcacattcttcaaggtcaaagtctagatcacagtctagttctcgatcaagatcaagatctcattctagaaagaagcgatacaggtctcgttccagaacatattcaaggtctcgtagtagagatcgtatgtattctagagattatcgtcgcgattacagaaataatagaggaatgagacgaccttatgggtacagaggaaggggtagagggtattatcaaggaggaggaggtagatatcatcgaggtggttatagacctgtctggaatagaaggcactctaggagtcctagacgaggtcgttcacgttccaggagtccaaaaagaagatccgtttcttctcaaagatccagaagcagatctcgccggtcatatagatcttctaggtctccaagatcatcctcttctcgttcttcatccccatatagcaaatctcctgtttctaaaagacgagggtctcaggaaaaacaaaccaaaaaagctgaaggggaaccccaagaagagagtccgttgaaaagtaaatcacaggaggaaccgaaagatacatttgaacatgacccatctgagtctatcgatgaatttaataagtcatcagccacatccggtgatatttggcctggcctttcagcttatgataatagtcctagatcaccccatagtccttcacctattgctacaccacctagtcagagttcatcttgctctgatgctcccatgctcagtacagttcactctgcaaaaaatactccttctcagcattcacattccattcagcatagtcctgaaaggtctgggtctggttctgttggaaatggatctagtcgatacagtccttctcagaatagtccaattcatcacatcccttcacgaagaagtcctgcaaagacaatcgcaccacagaatgctccaagagatgagtctaggggccgttcctcgttttatcctgatggtggagatcaggaaactgcaaagactgggaagttcttaaaaaggttcacagatgaagagtctagagtattcctgcttgataggggtaataccagggataaagaggcttcaaaagagaaaggatcagagaaagggagggcagagggagaatgggaagatcaggaagctctagattacttcagtgataaagagtctggaaaacaaaagtttaatgattcagaaggggatgacacagaggagacagaggattatagacagttcaggaagtcagtcctcgcagatcagggtaaaagttttgctactgcatctcaccggaatactgaggaggaaggactcaagtacaagtccaaagtttcactgaaaggcaatagagaaagtgatggatttagagaagaaaaaaattataaacttaaagagactggatatgtagtggaaaggcctagcactacaaaagataagcacaaagaagaagacaaaaattctgaaagaataacagtaaagaaagaaactcagtcacctgagcaggtaaagtctgaaaagctcaaagacctctttgattacagtccccctctacacaagaatctggatgcacgagaaaagtctaccttcagagaggaaagcccacttaggatcaaaatgatagcgagtgattctcaccgtcctgaagtcaaactcaaaatggcacctgttcctcttgatgattctaacagacctgcttccttgactaaagacaggctgcttgctagtacacttgtccattctgtcaagaaggagcaagaattccgatccatctttgaccacattaagttgccacaggccagcaaaagcacttcagagtcatttattcaacacattgtgtccttggttcatcatgttaaagagcaatacttcaagtcagctgcaatgaccctaaacgagcggttcacttcgtatcagaaagccactgaagaacatagtactcggcaaaagagccctgaaatacacaggagaattgacatctcaccaagtaccctgaggaagcatacccgtttagcaggggaagagagagtttttaaagaagaaaatcaaaagggagataaaaaattaaggtgtgactctgctgaccttcggcatgacattgatcgccgtagaaaagaaagaagtaaagaacggggagattccaagggctccagggaatccagtggatcaagaaagcaggaaaaaactccaaaagattacaaggaatacaaatcttacaaagatgacagtaaacataaaagagagcaagatcattctcgatcttcatcctcttcagcatcaccttcttctcccagttctcgagaagaaaaggagagtaagaaggaaagagaagaagaatttaaaactcaccatgaaatgaaagaatactcaggctttgcaggagttagccgaccacgaggaaccttttttcgaattagaggcagaggaagagccagaggagtttttgctgggacaaatactggtccaaacaactcaaatactacttttcaaaagagaccgaaggaagaggaatgggatccagaatataccccaaagagcaagaagtacttcttgcatgacgacagagatgatggtgtggattattgggccaaaagaggaagaggtcgtggtacttttcaacgtggcagagggcgctttaacttcaaaaaatcaggtagcagtcctaaatggactcatgacaaataccaaggggatgggattgttgaagatgaagaagagaccatggaaaataatgaagaaaagaaggacagacgcaaggaagaaaaggaataataaatatgaagtaagattacaacagagcagaacttgcacccaccattttttttacctgatttttgttttcaaataagaatgtaagcattttacttaaattttactgtttgcaagtagtctatagaaattttgttttaagtcttcaaatatcttgagaaatagtagactgtatgttgaaaattgtactgaaataaagtagaaaattgttacgtaccatatttgtaactatcaacttttaaaacttttaacgtttttgttacatgcattgtaattctgctttgtctataagatatggtcaagtacagctctgtgaaagttctgattctcttccttccctgtttgtcaatgttttattctgaagtaaacgttagctctacatataaatcctggaacagaaattgtttatagagactacactaattattttaactgtatacatctgtttaatttgaacacactacatcgtagggtgactgatttttgaagtataccacagacaaaaagttgttactatggtaaactaagctagtttaacacttgagcaaatgcttaagaaggaattaaaaaaaaaaagctttgccaatagctaaaaagtacaagctattaaaaatcagattgaaaagttttgagaaaatgttatttttactgaaagcaagcagtggcctataaagaacattcttaggagccttttctatttgcgttcaaaactgtgtgttctctttctattcctatttgatagtttgagtcatggtcttagatattagctatttgtgagaggaaactggtttgtaacaatactgcaaatagaaaccccatttctactgaacatcctagttttaaacagaagaaaaactgtaatcctggggttggtatgtaggaggtctatcctgcagaataagttgatacattagtacctgatttcatatcttacatatttatttgagctgaacattagtttgtagtgtaactattagtaaaaatagagaaacacagcatactgttcattaatagtattttaaaaaaattgtttttcaaatgtcaccaataaaagttttggcaggaagcttgttgcggcattgatctaacctttttcccccccatttcagttgcagtttttgtagaatggctttttctttttcctcttaagagttctattcttcaggtagataatttttcaaatgtgaattatcttttgtgtctatattgatagctcttaaaggagtgaaaatctaaaatagtaaatttcaatgttaagtgtctgctttatgggcatatataaaagtagacacatttcatttgttaatttagttgtgtgtgtgtgttaaaaggagctaatgcttattctgttaatgtaaacttttgaagatcttaagtgtattgctctttcatcttaaacactttcgaggatttgcagtgcgt

SEQ ID NO: 16 - RNA, 1001 nt, Homo sapiens
  source 1..1001: mol_type = mRNA
gaagattccagcaccctcccctaactccaggccagactctaaaggggagatctggatggcatctacttcgtatgactattgcagagtgcccatggaagacggggataagcgctgtaagcttctgctggggataggaattctggtgctcctgatcatcgtgattctgggggtgcccttgattatcttcaccatcaaggccaacagcgaggcctgccgggacggccttcgggcagtgatggagtgtcgcaatgtcacccatctcctgcaacaagagctgaccgaggcccagaagggctttcaggatgtggaggcccaggccgccacctgcaaccacactgtgatggccctaatggcttccctggatgcagagaaggcccaaggacaaaagaaagtggaggagcttgagggagagatcactacattaaaccataagcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtgagatcccaggaagctggcacatcttggaaggtccgtcctgctcggcttttcgcttgaacattcccttgatctcatcagttctgagcgggtcatggggcaacacggttagcggggagagcacggggtagccggagaagggcctctggagcaggtctggaggggccatggggaagtcctgggtgtggggacacagtcgggttgacccagggctgtctccctccagagcctccctccggacaatgagtcccccctcttgtctcccaccctgagattgggcatggggtgcggtgtggggggcatgtgctgcctgttgttatgggttttttttgcggggggggttgcttttttctggggtctttgagctccaaaaaataaacacttcctttgagggagagcacacctga

SEQ ID NO: 17-18 - skipped (2 entries)

SEQ ID NO: 19 - RNA, 1166 nt, Homo sapiens
  source 1..1166: mol_type = mRNA
ctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 20 - RNA, 1144 nt, Homo sapiens
  source 1..1144: mol_type = mRNA
agagacgacccgacccgctagaagactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggagcggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 21 - RNA, 21 nt, Human cytomegalovirus
  source 1..21: mol_type = genomic RNA
gcaagatctcgcacatcatgc

SEQ ID NO: 22 - RNA, 21 nt, Human cytomegalovirus
  source 1..21: mol_type = genomic RNA
gcgcatgtccagtctgtttaa

SEQ ID NO: 23 - RNA, 102 nt, Homo sapiens
  source 1..102: mol_type = other RNA
ggtcctctgactctcttcggtgacgggtattcttgggtggataatacggattacgttgttattgcttaagaatacgcgtagtcgaggagagtaccagcggca

SEQ ID NO: 24 - RNA, 81 nt, Homo sapiens
  source 1..81: mol_type = other RNA
cccattggcataaacccgtagatccgatcttgtggtgaagtggaccgcacaagctcgcttctatgggtctgtgtcagtgtg

SEQ ID NO: 25 - RNA, 976 nt, Homo sapiens
  source 1..976: mol_type = mRNA
gcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggctttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagacagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattg

SEQ ID NO: 26 - RNA, 1748 nt, Homo sapiens
  source 1..1748: mol_type = mRNA
gtgaggctgcgcttgcccggggcccgcgcccccctaccccggggaccgcccccgggccgcccgccccacttggcgcgccacttccgcgtgcatggccctgctgccccgagccctgagcgccggcgcgggaccgagctggcggcgggcggcgcgcgccttccgaggcttcctgctgcttctgcccgagcccgcggccctcacgcgcgccctctcccgtgccatggcctgcaggcaggagccgcagccgcagggcccgccgcccgctgctggcgccgtggcctcctatgactacctggtgatcgggggcggctcgggcgggctggccagcgcgcgcagggcggccgagctgggtgccagggccgccgtggtggagagccacaagctgggtggcacttgcgtgaatgttggatgtgtacccaaaaaggtaatgtggaacacagctgtccactctgaattcatgcatgatcatgctgattatggctttccaagttgtgagggtaaattcaattggcgtgttattaaggaaaagcgggatgcctatgtgagccgcctgaatgccatctatcaaaacaatctcaccaagtcccatatagaaatcatccgtggccatgcagccttcacgagtgatcccaagcccacaatagaggtcagtgggaaaaagtacaccgccccacacatcctgatcgccacaggtggtatgccctccacccctcatgagagccagatccccggtgccagcttaggaataaccagcgatggattttttcagctggaagaattgcccggccgcagcgtcattgttggtgcaggttacattgctgtggagatggcagggatcctgtcagccctgggttctaagacatcactgatgatacggcatgataaggtacttagaagttttgattcaatgatcagcaccaactgcacggaggagctggagaacgctggcgtggaggtgctgaagttctcccaggtcaaggaggttaaaaagactttgtcgggcttggaagtcagcatggttactgcagttcccggtaggctaccagtcatgaccatgattccagatgttgactgcctgctctgggccattgggcgggtcccgaataccaaggacctgagtttaaacaaactggggattcaaaccgatgacaagggtcatatcatcgtagacgaattccagaataccaacgtcaaaggcatctatgcagttggggatgtatgtggaaaagctcttcttactccagttgcaatagctgctggccgaaaacttgcccatcgactttttgaatataaggaagattccaaattagattataacaacatcccaactgtggtcttcagccacccccctattgggacagtgggactcacggaagatgaagccattcataaatatggaatagaaaatgtgaagacctattcaacgagctttaccccgatgtatcacgcagttaccaaaaggaaaacaaaatgtgtgatgaaaatggtctgtgctaacaaggaagaaaaggtggttgggatccatatgcagggacttgggtgtgatgaaatgctgcagggttttgctgttgcagtgaagatgggagcaacgaaggcagactttgacaacacagtcgccattcaccctacctcttcagaagagctggtcacacttcgttgagaaccaggagacacgtgtggcgggcagtgggacccatagatcttctgaaatgaaacaaataatcacattgaaaaaaaaaaaaaaaaaa

SEQ ID NO: 27 - skipped

SEQ ID NO: 28 - RNA, 21 nt, Homo sapiens
  source 1..21: mol_type = other RNA
tgattgtccaaacgcaattct

SEQ ID NO: 29 - RNA, 99 nt, Homo sapiens
  source 1..99: mol_type = other RNA
ccgatgtgtatcctcagctttgagaactgaattccatgggttgtgtcagtgtcagacctctgaaattcagttcttcagctgggatatctctgtcatcgt

SEQ ID NO: 30 - RNA, 85 nt, Homo sapiens
  source 1..85: mol_type = other RNA
ctgttctatggttagttttgcaggtttgcatccagctgtgtgatattctgctgtgcaaatccatgcaaaactgactgtggtagtg

SEQ ID NO: 31 - skipped

SEQ ID NO: 32 - RNA, 120 nt, Homo sapiens
  source 1..120: mol_type = other RNA
ggtacctgaaaagaagttgcccatgttattttcgctttatatgtgacgaaacaaacatggggtacctgaaaagaagttgcccatgttattttcgctttatatgtgacgaaacaaacatgg

SEQ ID NO: 33-37 - skipped (5 entries)